SEQUENCE LISTING (12 sequences)

SEQ ID NO: 1 - protein, 16 aa, Homo sapiens
KGYNYSYKSEMKVRPA

SEQ ID NO: 2 - protein, 19 aa, Homo sapiens
GGWTVFQRRVDGSVDFYRK

SEQ ID NO: 3 - protein, 27 aa, Homo sapiens
KGYNYSYKVSEMKFQRRVDGSVDFYRK

SEQ ID NO: 4 - protein, 18 aa, Homo sapiens
KGYKYSYKVSEMKVRPAK

SEQ ID NO: 5 - protein, 19 aa, Homo sapiens
GGWTVFQRRMDGSVDFYRK

SEQ ID NO: 6 - protein, 27 aa, Homo sapiens
KGYKYSYKGGWTVFQRRMDGSVDFYRK

SEQ ID NO: 7 - protein, 19 aa, Homo sapiens
GGWTVFQRRVDGSVDFYRC

SEQ ID NO: 8 - protein, 27 aa, Homo sapiens
KGYKYSYKVSEMKFQRRVDGSVDFYRC

SEQ ID NO: 9 - protein, 27 aa, Homo sapiens
KGYKYSYKVSEMKFQRRMDGSVDFYRK

SEQ ID NO: 10 - protein, 26 aa, Homo sapiens
KGYKYSYKGGWTVFQRRMDGSVDFYR

SEQ ID NO: 11 - protein, 27 aa, Homo sapiens
KTRWYSMKKTTMKVFQKRLVGSVDFKK

SEQ ID NO: 12 - protein, 19 aa, Homo sapiens
KTRWYSMKKTTMKIIPFNR

(19) United States
(12) Patent Application Publication
(10) Pub. No.: US 2011/0275573 A1
(43) Pub. Date: Nov. 10, 2011

(54) TISSUE ADHESIVE

(75) Inventors: Mazal Dahan, Mazkeret Batia (IL); Ascher Shmulewitz, Tel Aviv (IL)

(73) Assignee: METAMOREFIX LTD., Rehovot (IL)

(21) Appl. No.: 13/127,353

(22) PCT Filed: Nov. 3, 2009
(86) PCT No.: PCT/IL2009/001025

§ 371 Date: Jul. 27, 2011

Related U.S. Application Data
(60) Provisional application No. 61/193,176, filed on Nov. 3, 2008.

(51) Int. Cl. A61K 38/39; A61K 38/10; A61P 43/00; C07K 14/78; A61P 17/02; A61K 38/00; C07K 7/08

(52) U.S. Cl. 514/17.1; 530/326; 530/324; 514/19.1

(57) ABSTRACT

Provided is an adhesive for adhering tissues. The adhesive includes a scaffolding and one or more peptides or proteins capable of binding to cells of both the first and second tissues. In a embodiment, the proteins or peptides have an amino acid sequence that is a subsequence of a ficolin protein, for example, a human ficolin.

### FIELD OF THE INVENTION

This invention relates to surgical materials, and more specifically to surgical adhesives.

### BACKGROUND OF THE INVENTION

The following prior art publications are considered to be relevant for an understanding of the invention.
Blomback, B. 1996. Fibrinogen and Fibrin-Proteins with Complex Roles in Hemostasis and Thrombosis. In Thrombos. Res. Vol. 83, pp. 7-75.
Bailey, K., Bettelheim, F. R., Lorand L., et al., 1951. In Nature, vol. 167, pp 233.
Mosesson, M. W., Siebenlist, K. R., Armani, D. L., et al. 1989, In Proc. Natl. Acad. Sci. U.S.A., vol. 86, pp 1113.
D. A. Cheresh, S. A. Berliner, V. Vicente, C. M. Ruggeri: “Recognition of distinct adhesive sites on fibrinogen by related integrins on platelets and endothelial cells”, Cell 58, 1989, pp 945-953.
D. H. Farrell, P. Thiagarajan, D. W. Chung, E. W. Davie: “Role of finbrinogen \acute{α} and γ chain sites in platelet aggregation”, Proc. Nail. Acad. Sci. USA, 89, 1992, PP 10729-10732.
R. R. Hantgan, S. Endenburg, I. Cavero, G. Marguerie, A. Uzan, J. J. Sixma, P. G. de Groot: “Inhibition of platelet adhesion to Fibrin(ogen) in a flowing whole blood by RGD and fibrinogen γ chain carboxy terminal peptides” Thromb. Haemost. 68, 1992, pp 694-700.
B. Savage, E. Bottini, C. M. Ruggeri: “Interaction of integrin αIIb β with multiple fibrinogen domains during platelet adhesion”, J. Biol. Chem. 270, 1995, pp 28812-28817.

What is commonly referred to as blood clotting is based on a complex cascade of coagulation factors, ultimately resulting in the transformation of fibrinogen, a blood protein, into polymerized fibrin, making a clot. This process, also referred to as hemostasis, is the first of four distinct but overlapping phases related to wound healing. The clot formed in this phase is a cell adhering matrix which is required for the subsequent phases (including cell attraction and adhesion).

Fibrinogen protein chemistry and structure and its reactivity with thrombin has captured much of interest since the end of the 17^{th} century when the primary structural basis of a blood clot was described. Much information has since accumulated on this protein and its chemistry, as well as the functionality of each component in the clotting process, and the clot properties.

Relevant discoveries related to fibrinogen and fibrin polymerization include:

1. Fibrinogen consists of a dimer with the two subunits in an antiparallel orientation, where each subunit is composed of three polypeptide chains: Aα, Bβ and γ. The different chains are of molecular weight ranging from 48 to 70 kDa, and the MW of fibrinogen is 340 kDa
2. Thrombin is responsible for a two stage scission process of the fibrinogen: The release of peptide A (1^{st} stage) which allows the fibrin chains to be oriented, mainly in an end to end conformation, accompanied and followed by the release of peptide B, which allows specific folding that is essential for cross linking of the fibrin polymer (Factor XIII, Ca²⁺), creating hard and insoluble fibrin fibers.

The fibrin clot is commonly referred to as “glue” or “sealant” based on its ability to bind cells (mainly fibroblasts and endothelial cells). This capacity is a result of the clotting cascade, in which the N-terminal fibrinopeptide B is removed from the p chain of fibrinogen by thrombin. This leaves the N-terminal βB epitope 15-42 available for cell binding. The sequence 400-411 of the extreme C terminus of the γchain is also involved in hemostasis. It has been suggested in the past that the ROD epitope occurring in two locations on the αE chain are responsible for binding parenchymal cells, such as fibroblasts, endothelial and smooth muscle cells. Nevertheless, these RGD sites are poorly conserved in evolution. However, other sequences in fibrin(ogen) have been speculated by different groups to be responsible for this cell binding capacity based on various structural/functional criteria.

Fibrin sealants have gained increased popularity in many applications. The major applications of the natural sealant are as a topical agent for hemostasis and as an adhesive for tissue approximation, alone or combined with conventional suturing techniques. It is now used in a number of surgical specialties, including cardiovascular surgery, thoracic surgery, neurosurgery, plastic and re-constructive surgery and dental surgery.

Synthetic tissue adhesives are also known. The alkyl-cyanoacrylates were discovered in 1951, and filed for FDA approval in 1964. Protein based adhesives are also used (such as albumin glue with gluteraldehyde as crosslinker and gelatin based adhesive with resorcinol-formaldehyde complex). However, all of the synthetic glues suffer from two major problems, namely the slow degradation rate of the adhesives, thus not allowing the new fibroblasts to take over and fill the damaged area, and the degradation products, where formaldehyde is known to be one of them.

In recent years, several devices, based on another concept were FDA approved. These devices allow relatively rapid production of a cryoprecipitate (known to be fibrinogen rich, and also containing the factors VIII, XIII, von Willenbrand, and fibronectin) from a blood sample. The autologus cryoprecipitate is then reacted with thrombin from an exogenous source immediately before use to form an autologous sealant. These devices are very expensive and require guidance. Moreover, even when these conditions are fulfilled, the need to process the blood under critical conditions puts a barrier to their efficient use.

The standard practice of administration of the fibrin glue utilizes a dual-chamber syringe, which allows a 1:1 mixture of stabilized fibrinogen solution with a thrombin solution. The mixing action is the trigger for the clotting reaction that takes place in vivo. These kits are produced from blood plasma pools via long processes, based mostly on selective precipitation stages, leading to purified proteins (fibrinogen as the clot protein and thrombin). The raw material sources are limited and the down stream process decreases the efficiency and yield.

One of the risks related to the use of fibrin sealants/adhesives, although considered by the FDA to be biological compounds, stems from the fact that an active thrombin is released in the body. Potentially, there is a risk of fast diffusion of thrombin to react with endogenous fibrinogen rather than the injected exogenous fibrinogen. This could of course create thrombosis.

Another risk related to the use of these kits is the risk derived from using any plasma based product on a non-autologus basis: trans-contamination of one patient by another patient's blood. This may be minimized by close supervision of the regulation authorities, as well as by a wide range of quality assurance processes. The outcome of this situation is of course a product which is very expensive, not risk free, and based on limited sources.

The ficolins form a group of proteins having collagen- and fibrinogen-like domains. They were first identified as proteins that bind to TGF-β1. Three types of ficolin have been identified in humans: L-ficolin, H-ficolin and M-ficolin. A ficolin polypeptide consists of a small N-terminal domain, a collagen-like domain, a neck region, and a fibrinogen-like domain, which shows similarity to the C-terminal halves of the beta and gamma chains of fibrinogen. The collagen-like domain mediates the association of ficolin polypeptides into trimers, and the N-terminal domain contains cysteine residues which permit the covalent assembly of trimers into higher oligomers with a “bouquet-like” appearance. This supramolecular organization resembles that of the collectins, a group of C-type lectins which have a C-type CRD in place of the fibrinogen-like domain found in ficolins. Collectins and ficolins are also functionally similar. The collectin mannose binding protein (MBP) is a serum host defense protein in which the C-type CRDs recognize arrays of GlcNAc and mannose residues on pathogen surfaces. MBP initiates the lectin branch of the complement system via activation of MBP-associated proteases (MASPs), leading to elimination of the target pathogen. Two of the three human ficolins, ficolins L and H, are also serum proteins which bind to pathogen surfaces via interaction with carbohydrates (and probably with other molecules), and trigger complement activation though association with MASPs. Ficolin L also acts as an opsonin, promoting phagocytosis of pathogens by neutrophils. Ficolin L polymorphisms affect serum protein levels and sugar binding and may have pathophysiological implications. The third human ficolin, ficolin M, is found in secretory granules in neutrophils and monocytes, recognizes pathogens in a carbohydrate-dependent manner and activates complement via MASPs. Ficolin M may also act as a phagocytic receptor. Ficolins L and H are produced in the liver, in common with MBP, and ficolins M and H are produced in the lung, like the antimicrobial collectins SP-A and SP-D. Human ficolins and MBP also participate in the recognition and clearance of apoptotic cells. Two ficolins, A and B, are present in mouse. Ficolin B is found in the lysosomes of activated macrophages and is suggested to be the ortholog of ficolin M, but it appears that only ficolin A is associated with MASPs and can activate complement. The mouse ortholog of ficolin H is a pseudogene.

### DESCRIPTION OF THE INVENTION

The present invention is based upon the novel and unexpected finding that the protein ficolin is capable of binding to cell surfaces.

In one of its aspects, the invention provides an adhesive for adhering tissues. The adhesive of the invention comprises a biocompatible scaffolding and one or more proteins or peptides that are either bound to the scaffolding or are capable of being activated to bind to the scaffolding, where the peptides or proteins have amino acid sequences that are capable of binding to cells from the tissues to be adhered. In a preferred embodiment the peptides or proteins have amino acid sequences that are subsequences of ficolin, most preferably, human ficolin and are capable of binding the cells of the tissues to be adhered. The cells to be bound may be, for example, fibroblasts or endothelial cells.

In one preferred embodiment, the adhesive of the invention has an initial form that is essentially fluid, so that the adhesive can be applied to the tissue surfaces to be adhered. After application of the adhesive, the adhesive is made to undergo a process of curing or setting in which the adhesive solidifies. The curing or setting causes a change in the physical properties of the adhesive, to endow the adhesive with the desired mechanical strength, elasticity (tensile strength and elongation), viscosity, durability and degradation time. The curing or setting process may be initiated by exposure of the adhesive to an activator which may be a chemical activator, such as, a pH in a particular range or a cross-linker of the scaffolding. Exposure of the adhesive to a chemical activator may be performed using a double barrel syringe in which the adhesive and the activator are initially contained in different barrels. Alternatively, the curing process may be initiated by exposure of the adhesive to a form of energy such as an elevated temperature or electromagnetic radiation.

The proteins and peptides, when bound to the scaffolding, may be covalently bound, or non-covalently bound, for example, via hydrogen bonding, or hydrophobic bonding.

The adhesive of the invention is preferably biodegradable.

The scaffolding may be in the form of a bead, particle, membrane, fiber, oligomer or polymer of any molecular weight, capable of being chemically modified to bind to the peptides.

The scaffolding may be based on a naturally occurring polymer, existing in the human body, such as a polymer based on hyaluronic acid. In a preferred embodiment, the scaffolding comprises cross-linked hyaluronic acid or a salt thereof. The hyaluronic acid preferably has a molecular weight in the range of 0.7×10⁶ to 8×10⁶ Dalton. The scaffolding may be in the form of beads or particles or a membrane, and may be in any fowl of administration as required in any application. Methods for hyaluronic acid (HA) cross linking are well known in the art. The hyaluronic acid can be cross linked through each of the 3 functional groups attached to its backbone (the carboxylic group, the hydroxylic group, and the acetamido group.

The inventors have found that the following peptides, all having amino acid sequences that are subsequences of the amino acid sequence of human ficolin, may be used in the adhesive of the invention.
(a) the peptide, referred to herein as “C-Fic” and having the sequence KGYNYSYKSEMKVRPA;
(b) the peptide, referred to herein as “M-Fic” having the sequence GGWTVFQRRVDGSVDFYRK; and
(c) the peptide, referred to herein as “C-M-Fic” having the sequence KGYNYSYKVSEMKFQRRVDGSVDFYRK.
(d) the peptide, referred to herein as “C-Fic-a-K” having the sequence KGYKYSYKVSEMKVRPAK;
(e) the peptide, referred to herein as “M-Fic-K” having the sequence GGWTVFQRRMDGSVDFYRK;
(f) the peptide, referred to herein as “C-M-Fic2K” having the sequence KGYKYSYKGGWTVFQRRMDGSVDFYRK;
(g) the peptide, referred to herein as “M-Fic-S” having the sequence GGWTVFQRRVDGSVDFYRC; and
(h) the peptide, referred to herein as “CM-Fic-S” having the sequence KGYKYSYKVSEMKFQRRVDGSVDFYRC: and
(i) the peptide, referred to herein as “C-M-Fic-a-K” having the sequence KGYKYSYKVSEMKFQRRMDGSVDFYRK; and
(j) the peptide, referred to herein as “C-M-Fic2” having the sequence KGYKYSYKGGWTVFQRRMDGSVDFYR.

In another of its aspects, the invention provides a protein or peptide for use in the adhesive of the invention.

Thus, in one of its aspects, the invention provides an adhesive for adhering a first tissue to a second tissue comprising:
(a) a scaffolding
(b) one or more peptides or proteins selected from:
   (i) one or more peptides capable of binding to cells of both the first and second tissues; and
   (ii) a peptide or protein having a sequence homology of at least 70% with a peptide of (i) and capable of binding to cells in both of the first and second tissues;
the peptide either bound to the scaffolding or capable of being activated to become bound to the scaffolding; and
(c) a physiologically acceptable carrier.

The adhesive of the invention is preferably capable of curing or setting. The cells to which the protein or peptides bind may be, for example, fibroblasts and endothelial cells.

One or more of the proteins or peptides preferably may have an amino acid sequence that is a subsequence of a ficolin protein, most preferably a human ficolin. In particular one or more of the peptides or proteins may be selected from the group comprising at least:
(a) the peptide C-Fic having the sequence KGYNYSYKSEMKVRPA; and
(b) the peptide M-Fic having the sequence GGWTVFQRRVDGSVDFYRK; and
(c) the peptide C-M-Fic having the sequence KGYNYSYKVSEMKFQRRVDGSVDFYRK; and
(d) the peptide C-Fic-a-K having the sequence KGYKYSYKVSEMKVRPAK; and
(e) the peptide M-Fic-K having the sequence GGWTVFQRRMDGSVDFYRK; and
(f) the peptide, referred to herein as “M-Fic-S” having the sequence GGWTVFQRRVDGSVDFYRC; and
(g) the peptide, referred to herein as “CM-Fic-S” having the sequence KGYKYSYKVSEMKFQRRVDGSVDFYRC: and
the peptide C-M-Fic2K having the sequence KGYKYSYKGGWTVFQRRMDGSVDFYRK; and
(h) the peptide C-M-Fic-a-K having the sequence KGYKYSYKVSEMKFQRRMDGSVDFYRK; and
(i) the peptide C-M-Fic2 having the sequence KGYKYSYKGGWTVFQRRMDGSVDFYR.

The scaffolding of the adhesive preferably biodegradable, and may be in the form of beads, particles, fibers, oligomers or polymers. In one preferred embodiment, the scaffolding comprises hyaluronic acid or a salt thereof and the hyaluronic acid or acid salt is preferably cross-linked. Typically, the hyaluronic acid has an average molecular weight in the range of 0.7×106 to 3×106 Dalton.

In one preferred embodiment, the adhesive has a form suitable for injection.

In another of its aspects, the invention provides a protein or peptide for use in the adhesive of the invention. In one preferred embodiment of this aspect of the invention, the protein or peptide is selected from the group comprising:
(a) the peptide C-Fic having the sequence KGYNYSYKSEMKVRPA; and
(b) the peptide M-Fic having the sequence GGWTVFQRRVDGSVDFYRK; and
(c) the peptide C-M-Fic having the sequence KGYNYSYKVSEMKFQRRVDGSVDFYRK; and
(d) the peptide C-Fic-a-K having the sequence KGYKYSYKVSEMKVRPAK; and
(e) the peptide M-Fic-K having the sequence GGWTVFQRRMDGSVDFYRK; and
(f) the peptide, referred to herein as “M-Fic-S” having the sequence GGWTVFQRRVDGSVDFYRC; and
(g) the peptide, referred to herein as “CM-Fic-S” having the sequence KGYKYSYKVSEMKFQRRVDGSVDFYRC: and
(h) the peptide C-M-Fic2K having the sequence KGYKYSYKGGWTVFQRRMDGSVDFYRK; and
(i) the peptide C-M-Fic-a-K having the sequence KGYKYSYKVSEMKFQRRMDGSVDFYRK; and
(j) the peptide C-M-Fic2 having the sequence KGYKYSYKGGWTVFQRRMDGSVDFYR.

In another of its aspects, the invention provides use of the adhesive of the invention for adhering tissues. In particular, the invention provides use of the adhesive of the invention for adhering connective tissue.

The invention also provides use of a protein or a peptide according to Claim 17 for the preparation of an adhesive of the invention.

The invention further provides a method for adhering two or more tissues comprising applying to one or more of the tissues an adhesive of the invention and adjoining the two or more tissues. In particular, the invention provides adhering two or more tissues where one or more of the tissues is connective tissue. The method may be carried out by administering the adhesive by injection.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1 shows cell binding of FF1 to PreC and C-Fic-aK, 6 mg peptide/ml;

FIG. 2 shows cell binding of FF1 to PreC, C-M-Fic, and C-M-Fic-a-K at a concentration of 6 mg peptide/ml, and the peptide C-M-Fic at a concentration of 12 mg peptide'

FIG. 3a shows cell binding of FF1 to C-M-Fic2;

FIG. 3b shows the activity of M-fic-K, and M-Fic in comparison with the positive control PreC-gamma, 6 mg peptide/ml;

FIG. 4 shows cell binding of FF1 to C-M-Fic, M-Fic, and C-Fic over a period of 24 hours (FIG. 4a) and 150 hours (FIG. 4b);

FIG. 5 shows cell binding of BAEC to C-M-Fic, M-Fic, and C-Fic over a period of 24 hours (FIG. 5a) and 150 hours (FIG. 5b);

FIG. 6 shows cell binding of FF1 to C-M-Fic, M-Fic, and C-Fic at 6 mg peptide/ml over a period of 24 hours (FIG. 6a) and 150 hours (FIG. 6b);

FIG. 7 shows cell binding of BAEC to C-M-Fic, and M-Fic, C-Fic at 6 mg peptide/ml a period of 24 hours (FIG. 7a) and 150 hours (FIG. 7b);

FIG. 8 shows the dose responses of cell binding of FF1 to sepharose beads coated with the peptides C-Fic-6 and C-Fic-12 (FIG. 8a), the peptides M-Fic-6 and M-Fic-12 (FIG. 8b); and the peptides C-M-Fic-6 and C-M-Fic-12 (FIG. 8c);

FIG. 9 shows the dose responses of cell binding of BAEC to the peptides C-Fic-6 and C-Fic-12 (FIG. 9a), the peptides M-Fic-6 and M-Fic-12 (FIG. 9b); and the peptides C-M-Fic-6 and C-M-Fic-12 (FIG. 9c) the various peptides;

FIG. 10 shows Nomarsky optic microscopy of peptide coated beads following attachment to FF1 after 3 days of incubation, (×100) (left panel: C-Fic, middle panel: C-M Fic, right panel: M-C-Fic);

FIG. 11 shows Nomarsky optic microscopy of peptide coated beads and their attachment to FF1 after 3 weeks of incubation (×100) (Upper left panel: blank, upper right panel: C-Fic, lower left panel: M-fic, lower right panel: C-M-Fic);

FIG. 12 shows Nomarsky optic microscopy of peptide coated beads and their attachment to endothelial cells (BAEC) following 3 weeks of incubation, (×100) (Upper left panel: blank, upper right panel: C-Fic, lower left panel: M-fic, lower right panel C-M-Fic);

FIG. 13 shows the toxicity of C-Fic, M-Fic, and M-C-Fic to FF1 cells (FIG. 13a) and BAEC (FIG. 13b) over a wide range of peptide concentration after 48 hours;

FIG. 14 shows the toxicity of the various peptides to FF1 cells (FIG. 14a) and BAEC (FIG. 14b) after 5 days of incubation; and

FIG. 15 shows the attachment of FF1 cells in suspension to the coated sepharose beads.

### EXAMPLES

### Materials and Methods

### HA Beads Formation:

### Solutions:

a. 5 mg/ml sodium hyaluronate (Na-HA,BTG Ltd) in PBS, pH adjusted to 4.8 with 0.1N hydrochloric acid (HCl).
b. Solid AAD (adipic acid dihydrazide (Sigma)
c. Solid EDC (1-Ethyl-3[3-dimethylaminopropyl]carbodiimide hydrochloride) from Sigma.
d. Solid N-hydroxysulfosuccinimide (sulfo-NHS,Sigma)
e. Corn oil (Sigma)

0.08 g of solid EDC and 0.15 g of Sulfo-NHS were added to 200 ml of 0.5% Na-HA, and mixed well for 30 Minutes in room temperature. pH was adjusted to 4.8, and 0.8 g solid AAD were added. Reaction solution was stirred at room temperature for 2 hours, and pH was than adjusted to 7 with 1N NaOH, to stop the reaction.
10 ml of the cross linked gel spread on a polyethylene membrane and left to air dry as a film
4 ml of the cross linked Na-HA gel, in 4 eppendorfs were dried in a Speed Vac apparatus (centrifuged under vacuum at elevated temperature, 60° C.).
185 ml of the gel were slowly poured into heated corn oil (60° C.), while controlled stirring (start 400-450 rpm). Turbidity indicates formation of reversed emulsion. After 12 hours stirring, 2 phases were formed and the beads were separated by filtration, washed with ethanol or hexane.
The following beads size distribution was obtained (using a vibrating mesh system, Frisch):

| Particle Size (μ) | % W/w |
|---|---|
| Larger than 125 | 69.5 |
| Smaller than 125 and larger than 100 | 6.1 |
| Smaller than 100 | 22.3 |
| Weight Loss (on mesh) | 2.1 |

Attachment of Peptides to Sepharose Beads as Carriers (for In Vitro Assays)

Solutions:
a. 1 mM HCl solution for activation of CNBr Sepharose beads at 4° C.
b. Coupling buffer containing 0.1 M NaHCO₃ and 0.5 N NaCl adjusted to pH 8.3.
c. Blocking solution to block remaining active groups on the SEPHAROSE BEADS gel containing 0.2M Glycine, pH 8.0 at 4° C.
d. Acetate buffer containing 0.1 M Acetate buffer pH 4.0, 0.5 M NaCl
Peptide solutions: Table 1 shows the peptides that were used.

TABLE 1

| Peptide | Sequence |
|---|---|
| C-Fic | KGYNYSYKSEMKVRPAK |
| M-Fic | GGWTVFQRRVDGSVDFYRK |
| C-M-Fic | KGYNYSYKVSEMKFQRRVDGSVDFYRK |
| C-Fic-a-K | KGYKYSYKVSEMKVRPAK |
| M-Fic-K | GGWTVFQRRMDGSVDFYRK |
| CM-Fic-a-K | KGYKYSYKVSEMKFQRRMDGSVDFYRK |
| CM-Fic-2 | KGYKYSYKGGWTVFQRRMDGSVDFYR |
| CM-Fic-2K | KGYKYSYKGGWTVFQRRMDGSVDFYRK |
| Controls | |
| C-M-g | KTRWYSMKKTTMKVFQKRLVGSVDFKK |
| preCγ | KTRWYSMKKTTMKIIPFNR |

Peptide solutions (2 mg/ml) were prepared in coupling buffer. For insoluble peptides, the dry peptides were pre-dissolved in 50 μl DMSO before the addition of the coupling buffer. The exact concentration of the peptide solutions was determined spectrophotometrically at OD₂₈₀.

Coupling Peptides to Sepharose Beads

100 mg of dried CNBr-Sepharose Beads (yields 350 μl of swollen Sepharose Beads-gel) was introduced into a disposable polystyrene mini-column (FIG. 1), appropriate for the preparation of a 0.5-2 ml gel filtration column (Pierce, Prod #29920). The lower part of the columns was blocked by a thick glass filter and a stopper.

3-4 ml total volume of HCl solution (1 mM) was added to the column, and the liquid was forced to flow under vacuum pressure through the gel. This process was repeated 5 times. The final aliquot of HCl was aspirated until cracks appeared in the gel cake. 2-3 ml aliquots of coupling buffer were immediately added to the washed beads and aspirated. This step was repeated 4-5 times. The high pH hydrolyzes and opens the active groups.

Peptide Binding

A peptide solution in coupling buffer prepared as above was immediately added to the activated Sepharose beads Sepharose beads were used to fixate the peptides for all in-vitro assays in the column to a final binding of 6 mg peptide per ml gel. The column was closed and shaken overnight at 4° C. very gently to avoid mechanical breakage of the beads. The bottom cap of the column was removed and the solution of ligand/buffer was collected as the Sepharose Beads settled on the filter. The OD₂₈₀ of the collected buffer was checked to determine the concentration of uncoupled peptide. The gel was washed/aspirated with 5 volumes (˜2 ml) of coupling buffer and gently mixed. Any remaining active groups on the gel were blocked with blocking buffer for 2 hours at room temperature or overnight at 4° C. The Sepharose beads gel with peptides was then washed by 2 cycles each of 5 gel volumes of:
a. 0.1 M Acetate buffer
b. Coupling buffer
c. Storing buffer: azide-coupling buffer: coupling buffer containing 0.1% NaN₃ (final concentration)

The Sepharose Beads were stored for till use at 4-8° C. Prior to use, the beads were in an eppendorf tube 4 times for 3 min each with PBS or medium.

### Cell Attachment Assay in Monolayers

The tested peptides (Table 1) were coupled to Sepharose Beads at two concentrations: 6 and 12 mg/ml Sepharose Beads. Sepharose Beads that underwent the coupling procedure without peptide addition (“naked Sepharose Beads”) served as a negative control (referred to herein as “Sepharose Beads-blank”). A cell binding assay was performed with 2 normal cell types: bovine aortal endothelial cells (BAEC) and human foreskin fibroblasts (FF1). Sepharose beads were added to 12 well culture plates with sub-confluent monolayer of the cultured cells (100-300 beads were added to each well). The fraction of Sepharose beads attached to the monolayer was determined at different times.

Cell Attachment Assay in Cell Suspensions.

Attachment of FF1 in suspension to peptides bound to sepharose beads was followed using the MTS assay. The MTS assay uses tetrazolium, which is taken up by viable cells and converted into formazan, which has a light absorption peak at 492 nm. The light absorbance at 492 (OD₄₉₂) is directly proportional to the number of the living cells attached to the beads. Calibration curves were used to transform the OD₄₉₂ readings into cell number,

Results

FIG. 1 shows cell binding of FF1 to PreC and C-Fic-aK, 6 mg peptide/ml. The results indicate a similar rate of cell binding of the tested peptide compared with the positive control. Both peptides reach 100% cell binding within less than 24 hours.

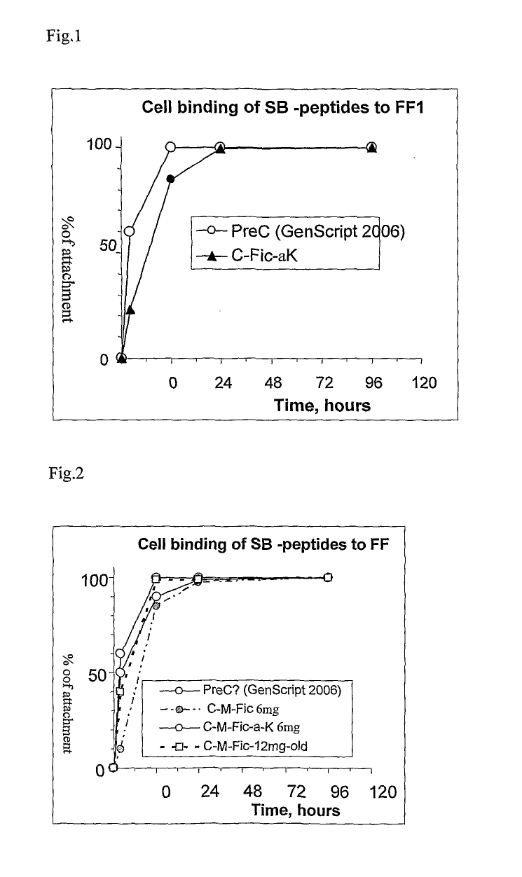

FIG. 2 shows cell binding of FF1 to PreC, C-M-Fic, and, C-M-Fic-a-K at a concentration of 6 mg peptide/ml, and the peptide C-M-Fic at a concentration of 12 mg peptide. All of the peptides tested were bound the cells with a similar kinetics as the positive control protein PreC. The higher concentration of the peptide C-M-Fic (12 mg/ml) showed a slightly faster cell binding compared to the lower concentration (6 mg/ml). Modifying the peptide C-M-Fic to produce the peptide C-M-Fic-aK accelerated the kinetics of cell binding.

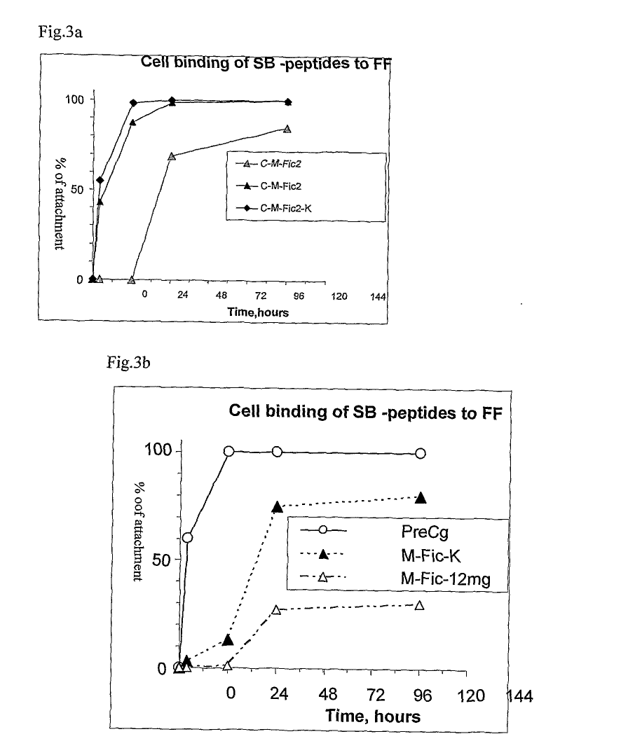

FIG. 3a shows cell binding of FF1 to C-M-Fic2. C-M-Fic2-K differs from C-M-Fic2 by the addition of a lysine group. Both C-M-Fic2 and C-M-Fic2-K show a good kinetic profile of cell binding (fast attachment), and reach 100% binding. Nevertheless, an addition of a FITC group (highly hydrophobic) inhibits both the rate and probably the total extent of cell binding.

FIG. 3b shows the activity of M-fic-K, and M-Fic in comparison with the positive control PreC-gamma, 6 mg peptide/ml. As observed also in the comparison of C-M-Fic2 and C-M-Fic2-K (FIG. 3a), the addition of a lysine group resulted a dramatic change in activity, although M-Fic was initially inferior to the positive control.

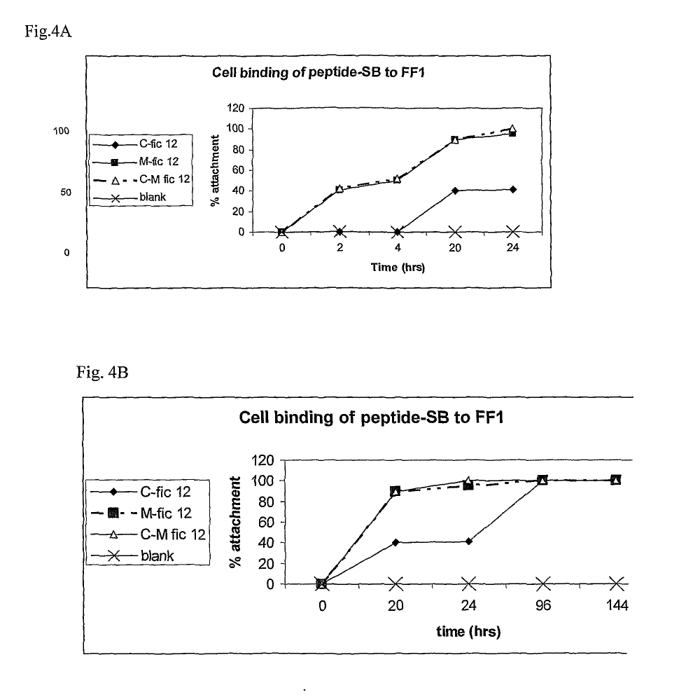

FIG. 4 shows cell binding of FF1 to C-M-Fic, M-Fic, C-Fic and blank (uncoated beads), at 12 mg peptide/ml concentration, over 24 hours (FIG. 4a). Blank sepharose beads do not have any cell binding capability, therefore confirming the peptides as the source of binding. Various kinetic profiles of different peptides can be shown, already within the first 12 hours. Some show a lag time before cell binding, whereas others show an immediate response. The peptides also differ in their maximal capacity for binding (some reach 100% and some bind about 50% of the cells). FIG. 4b shows the same list of peptides after 150 hours. Of particular interest is the observation that even the slower peptide (C-Fic) reaches 100% cell binding after a period of time,

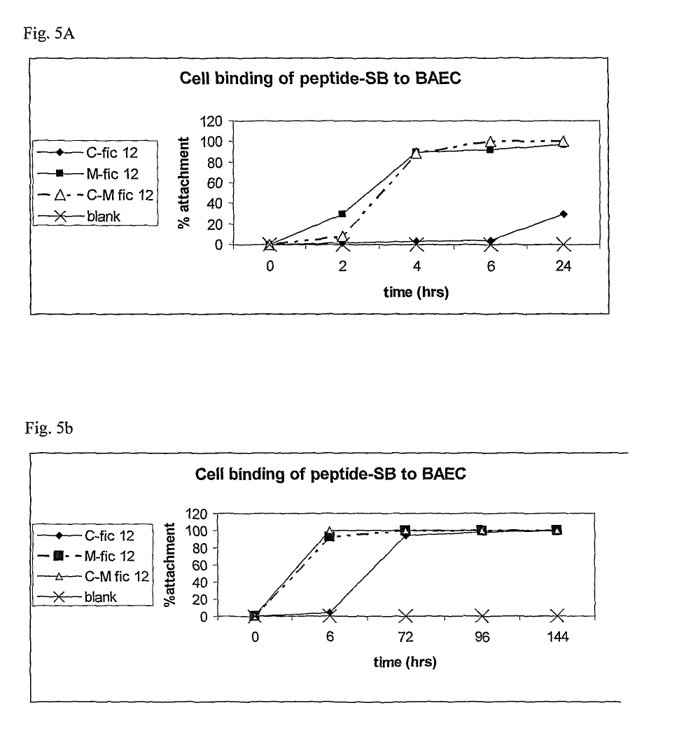

FIG. 5 shows cell binding of BAEC to C-M-Fic, M-Fic, C-Fic and blank, 12 mg peptide/ml concentration, over 24 hours (FIG. 5a). As in the previous test group of peptides—various profiles are detected. FIG. 5b follows the BAEC binding after 150 hours, where all peptides reach complete cell binding.

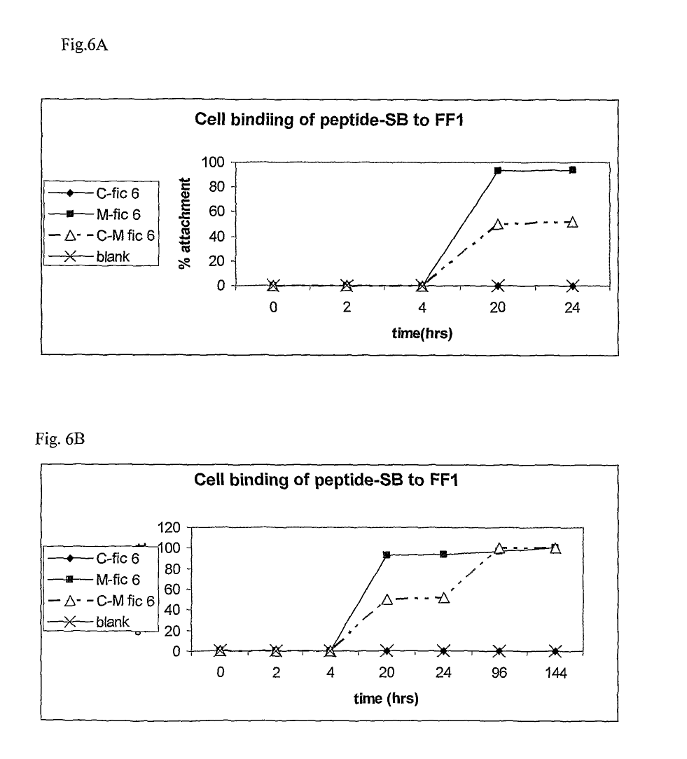

FIG. 6 shows cell binding of FF1 to C-M-Fic, M-Fic, C-Fic and blank (6a after 24 hours, and 6b after 150 hours), at 6 mg peptide/ml concentration, over 24 hours (FIG. 6a). Comparing the results to FIG. 4, an evident decrease in binding rate is observed. This is a dose response test, allowing differentiation between the active peptides at 12 mg/ml. C-fic looses its activity, C-M-fic is strongly affected by the lower dose, whereas M-fic seems to retain most of its activity.

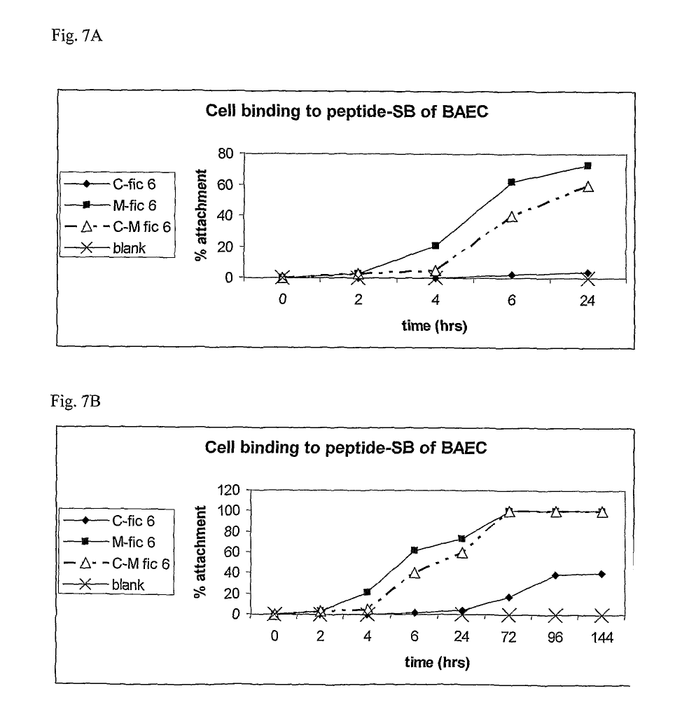

FIG. 7 shows cell binding of BAEC to C-M-Fic, M-Fic, C-Fic and blank, 6 mg peptide/ml, over 24 hours (FIG. 7a) and 150 hours (FIG. 7b). Compared with the 12 mg/ml results (FIG. 5) the BAEC seem to be more sensitive to the dose decrease. This is evident both in the short term and in the longer term.

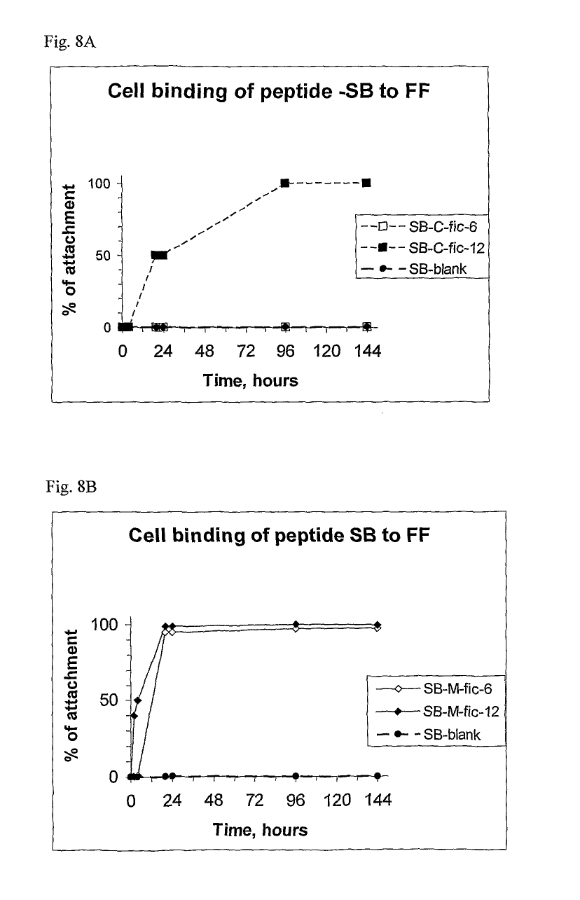

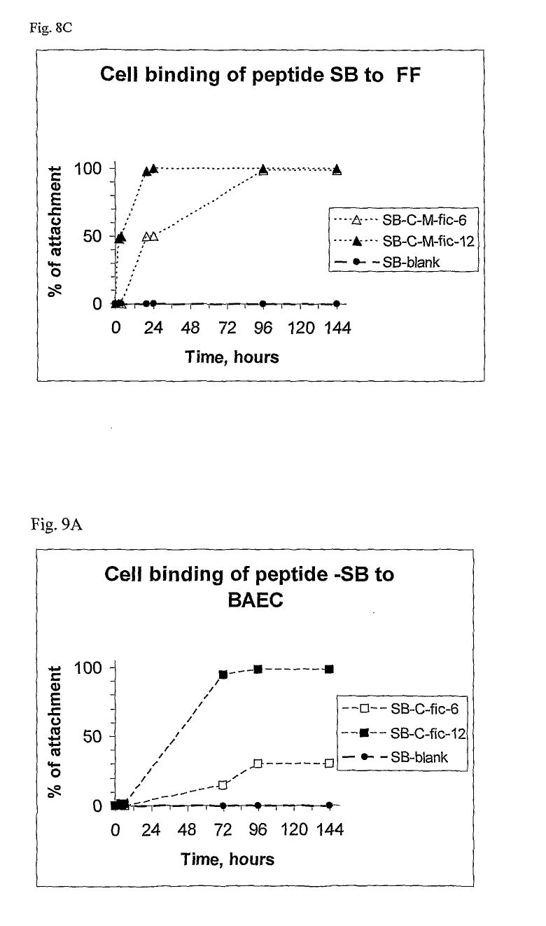

FIG. 8 shows the dose responses of cell binding of FF1 to sepharose beads coated with one of the peptides, or with no coat (SB-blank). C-Fic (FIG. 8a), has no reactivity at 6 mg/ml. M-Fic (FIG. 8b,) retains its activity at the lower concentration. Cell binding of FF1 to C-M-Fic (FIG. 8c) is strongly affected by the decrease and reaches 100% cell binding only after 96 hrs (compared to 20 hrs in the higher dose). The results presented in FIG. 8 are over 150 hours.

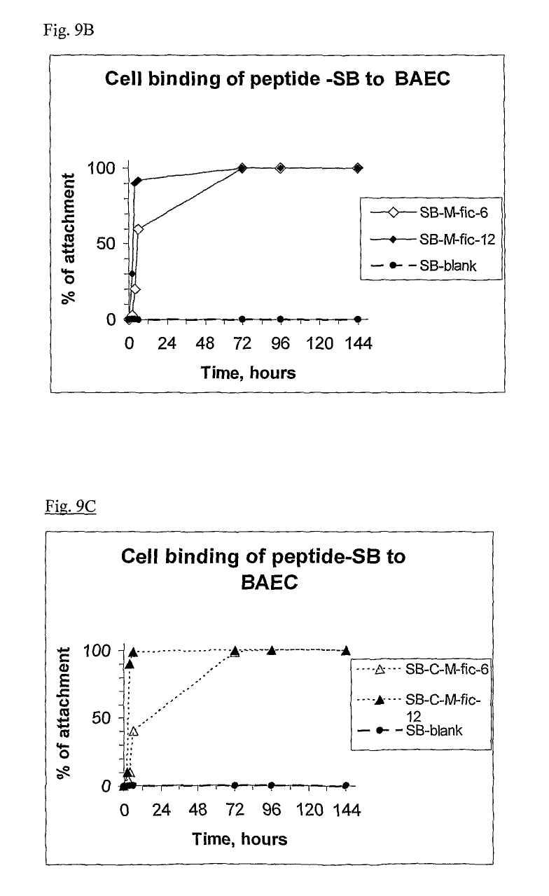

FIG. 9 shows the dose responses of cell binding of BAEC to each peptide. C-Fic (FIG. 9a), completely looses reactivity at 6 mg/ml. There is a slight decrease in M-Fic binding rate at the lower concentration (FIG. 9b), and cell binding of BAEC to C-M-Fic (FIG. 9c) has a slower rate at the lower concentration.

The following conclusions could be drawn:

1. Sepharose Beads alone did not attach to the cell monolayer of either cell type. Therefore the reactivity of cell binding shown in all the other experiments can be attributed to the peptides.

2. At a concentration of 12 mg peptide/ml, all of the tested peptides reach a 100% cell binding at a relatively short time, both with FF1 and BAEC.

3. In general BAEC interacted with the peptides faster than FF1.

4. A dose response approach was tested, in order to fine tune the screening between the various peptides.

5. Even at a concentration of 6 mg/ml, M-fic reached saturated cell binding within 24 hrs. C-M-fic reached saturated cell binding within 96 hrs. At this concentration, C-fic was inactive. For BAEC, similar results were obtained, but for these cells, C-fic also showed some activity.

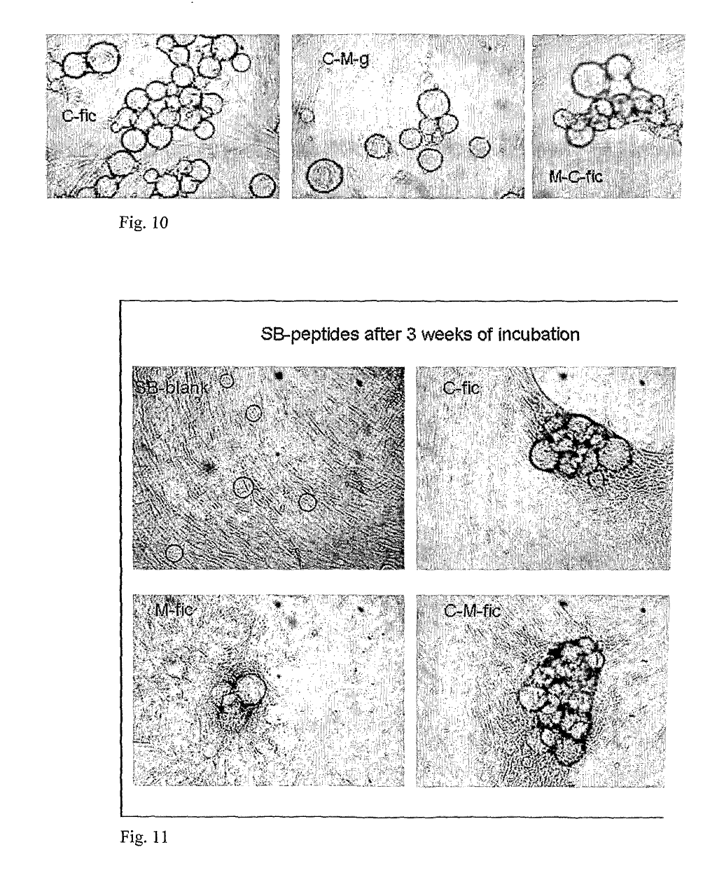

FIG. 10 shows Nomarsky optic microscopy of peptide coated beads following attachment to FF1 after 3 days of incubation, (×100) (left panel, C-Fic, middle panel: C-M Fic, right panel: M-C-Fic). Note the aggregates of cells and beads formed with the peptides M-fic, C-M-Fic. FIG. 11 shows Nomarsky optic microscopy of peptide coated beads and their attachment to FF1 after 3 weeks of incubation, (×100). (Upper left panel: blank, upper right panel: C-Fic, lower left panel: M-fic, lower right panel: C-M-Fic). The same response is seen as in FIG. 10, but in places where the cells aggregated, the aggregate was more pronounced even with less active peptides.

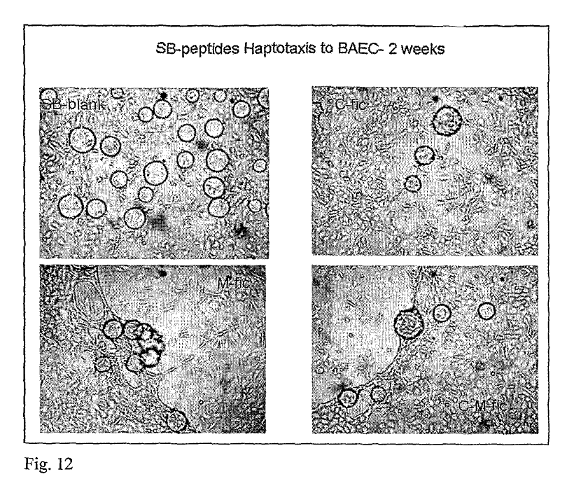

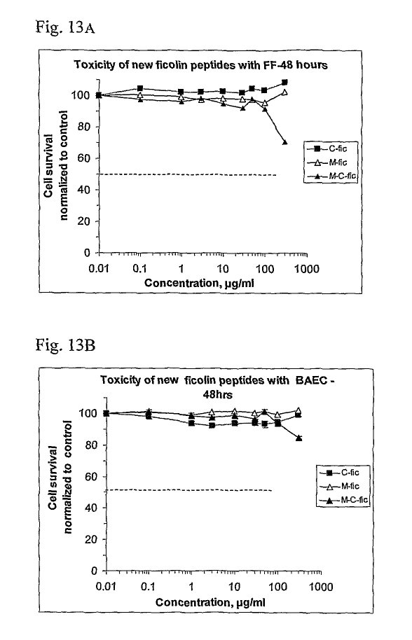

FIG. 12 shows Nomarsky optic microscopy of peptide coated beads and their attachment to endothelial cells (BAEC) following 3 weeks of incubation (×100). (Upper left panel: blank, upper right panel: C-Fic, lower left panel: M-fic, lower right panel: C-M-Fic). Note the formation of small tubes associated with sepharose beads bound to M-fic and CM-fic.

At longer incubation times, the peptides further immobilized FF1 to the beads forming stable bead-cell aggregates. This attachment increased with time without loss of the effect, as shown in FIGS. 10 and 11 for the different peptides tested. Sepharose beads coated with the active peptides fowl on the plate 3-D structures at a high cell density.

With BAEC, the cells were mobilized to the same peptides. In FIG. 12 it is evident that the sepharose beads coated with the active peptides induced aggregates and tube like structures.

M-fic was of the highest activity and C-M-Fic was also active.

Toxicity Assay for the Different Peptides:

A toxicity assay was done to determine the toxicity of the peptides tested to either one of the cell lines used. 15×10³ cells (FF1 or BAEC) were seeded in 96 well plastic plates. After overnight incubation, increasing concentrations of peptides in the range of 0.1-300 μg/ml were added to the wells. Cell survival was checked by the MTS assay after 2 and 5 days and normalized to the cell number of the controls (no peptide).

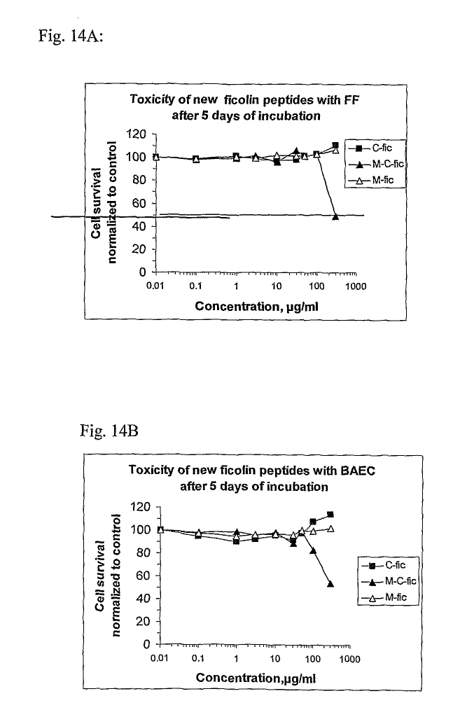

FIG. 13 shows the toxicity of C-Fic, M-Fic, and M-C-Fic to FF1 cells (FIG. 13a) and BAEC (FIG. 13b) over a wide range of peptide concentrations after 48 hours. FIG. 14 shows the toxicity of the various peptides to FF1 cells (FIG. 14a) and BAEC (FIG. 14b) after 5 days of incubation.

Attachment of Cells in Suspension to Peptide—Bead Constructs.

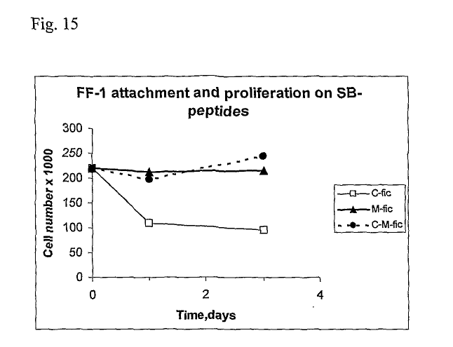

FIG. 15 shows the attachment of FF1 cells in suspension to the coated sepharose beads. For C-fic and CM-fic coated beads, cell attachment was stable for at least three days. For C-M-fic, an increase in cell number is detected after the first day, indicating a proliferation of cells. For C-fic coated beads, cell attachment decreased during the first day and then remained stable.

### CONCLUSIONS

1. In general, the peptides at the lower peptide concentration of 6 ml/mg attached at a slower rate to either cell type, in comparison to the higher concentration (12 mg/ml).

2. C-fic at a concentration of 6 mg peptide/ml did not attach to FF1 or to BAEC. The extent of cell binding was about 30% after 96 hours of incubation.

3. M-fic and C-M-fic reached saturated attachment after 72 hours of incubation.

4. Most of the peptides were not toxic in the wide range of concentrations tested. At very high concentrations (>100 μg/ml), some enhancement of proliferation was observed for some of the peptides.

5. C-M-fic showed some minor toxicity at very high concentrations (>100 μg/ml) for both cell types and prolonged exposure (5 days). At high concentrations of this peptide, cell survival, normalized to controls, was about 50%.

1.-21. (canceled)

22. An adhesive for adhering a first tissue to a second tissue, comprising:
(a) a scaffolding
(b) one or more peptides or proteins selected from:
(i) one or more peptides capable of binding to cells of both the first and second tissues; and
(ii) a peptide or protein having a sequence homology of at least 70% with a peptide of (i) and capable of binding to cells in both of the first and second tissues;
the peptide either bound to the scaffolding or capable of being activated to become bound to the scaffolding; and
(c) a physiologically acceptable carrier.

23. The adhesive according to claim 22, wherein one or more of the proteins or peptides has an amino acid sequence that is a subsequence of a ficolin protein.

24. The adhesive according to claim 23, wherein the ficolin protein is a human ficolin protein.

25. The pharmaceutical composition according to claim 23, wherein one or more of the peptides or proteins is selected from the group consisting of
(a) the peptide C-Fic having the sequence KGYNYSYKSEMKVRPA,
(b) the peptide M-Fic having the sequence GGWTVFQRRVDGSVDFYRK,
(c) the peptide C-M-Fic having the sequence KGYNYSYKVSEMKFQRRVDGSVDFYRK,
(d) the peptide C-Fic-a-K having the sequence KGYKYSYKVSEMKVRPAK,
(e) the peptide M-Fic-K having the sequence GGWTVFQRRMDGSVDFYRK,
(f) the peptide, referred to herein as “M-Fic-S” having the sequence GGWTVFQRRVDGSVDFYRC,
(g) the peptide, referred to herein as “CM-Fic-S” having the sequence KGYKYSYKVSEMKFQRRVDGSVDFYRC,
(h) the peptide C-M-Fic2K having the sequence KGYKYSYKGGWTVFQRRMDGSVDFYRK,
(i) the peptide C-M-Fic-a-K having the sequence KGYKYSYKVSEMKFQRRMDGSVDFYRK, and
(j) the peptide C-M-Fic2 having the sequence KGYKYSYKGGWTVFQRRMDGSVDFYR.

26. The adhesive according to claim 22, being capable of curing or setting.

27. The adhesive according to claim 22, wherein the cells to which the protein or peptides bind are selected from fibroblasts and endothelial cells.

28. The adhesive according to claim 22, wherein the scaffolding is biodegradable.

29. The adhesive according to claim 22, wherein the scaffolding is in the form of beads, particles, fibers, oligomers or polymers.

30. The adhesive according to claim 22, wherein the scaffolding comprises hyaluronic acid or a salt thereof.

31. The adhesive according to claim 30, wherein the hyaluronic acid or acid salt is cross-linked.

32. The adhesive according to claim 30, wherein the hyaluronic acid has an average molecular weight in the range of from 0.7×10⁶ to 3×10⁶ Dalton.

33. The adhesive according to claim 22, wherein the scaffolding is crosslinkable.

34. The adhesive according to claim 22, having a form suitable for injection.

35. A protein or peptide for use in the adhesive according to any claim 22.

36. The protein or peptide according to claim 35, selected from the group consisting of
(a) the peptide C-Fic having the sequence KGYNYSYKSEMKVRPA,
(b) the peptide M-Fic having the sequence GGWTVFQRRVDGSVDFYRK,
(c) the peptide C-M-Fic having the sequence KGYNYSYKVSEMKFQRRVDGSVDFYRK,
(d) the peptide C-Fic-a-K having the sequence KGYKYSYKVSEMKVRPAK,
(e) the peptide M-Fic-K having the sequence GGWTVFQRRMDGSVDFYRK,
(f) the peptide, referred to herein as “M-Fic-S” having the sequence GGWTVFQRRVDGSVDFYRC,
(g) the peptide, referred to herein as “CM-Fic-S” having the sequence KGYKYSYKVSEMKFQRRVDGSVDFYRC,
(h) the peptide C-M-Fic2K having the sequence KGYKYSYKGGWTVFQRRMDGSVDFYRK,
(i) the peptide C-M-Fic-a-K having the sequence KGYKYSYKVSEMKFQRRMDGSVDFYRK, and
(j) the peptide C-M-Fic2 having the sequence KGYKYSYKGGWTVFQRRMDGSVDFYR.

37. A method for adhering two or more tissues comprising applying to one or more of the tissues an adhesive according to claim 22, and adjoining the two or more tissues.

38. The method according to claim 37, wherein one or more of the tissues is connective tissue.

39. The method according to claim 37, wherein the composition is administered by injection.
